(19) United States
(12) Patent Application Publication
(10) Pub. No.: US 2025/0098947 A1
(43) Pub. Date: Mar. 27, 2025

(54) AN ARTICULATING CANNULA

(71) Applicant: SSI IP HOLDINGS INC., Fort Lauderdale, FL (US)

(72) Inventors: Sudhir Prem SRIVASTAVA, Gurugram, Haryana (IN); Vishwajyoti Pascual SRIVASTAVA, Gurugram, Haryana (IN); Yogesh SINGH, Gurugram, Haryana (IN)

(21) Appl. No.: 18/289,768

(22) PCT Filed: Jun. 27, 2023
(86) PCT No.: PCT/IN2023/050624

§ 371 Date: Nov. 7, 2023

(30) Foreign Application Priority Data
Jun. 28, 2022 (IN) 202211036953

(51) Int. Cl. A61B 1/008; A61B 1/005

(52) U.S. Cl. CPC A61B 1/008; A61B 1/0057

(57) ABSTRACT

The application provides an articulating cannula (300) comprising a proximal end (302) and a distal end (304) connected with a hollow tubular shaft (306). The distal end (304) is configured to articulate between a first position (310) and a second position (312). The shaft (306) is segmented into a first shaft (414) and a second shaft (316) which are secured by a flexible material. The proximal end (302) comprises of a housing (308) having diameter substantially greater than the diameter of the shaft (306) and configured to allow insertion of an articulating surgical instrument (318). The second shaft (316) comprises of an outer layer (320) and an inner layer (322) of the flexible material with a compression spring (324) in between, the second shaft (316) is configured to facilitate the articulation of the distal end (304) and to prevent collapse of second shaft (316) at the distal end (304). The spring (324) is configured to maintain a constant cross section of the second shaft (316) with or without articulation.

### CROSS-REFERENCE TO RELATED APPLICATIONS AND PRIORITY

This application claims priority from International Application No. PCT/IN2023/050624, filed on Jun. 27, 2023, which application claims priority from Indian patent application Ser. No. 20/221,1036953, filed on Jun. 28, 2022.

### TECHNICAL FIELD

The present disclosure generally relates to a medical device for insertion into tissue during minimal invasive surgical procedure, and more particularly, the disclosure relates to a cannula with an articulating distal end.

### BACKGROUND

This section is intended to introduce the reader to various aspects of art that may be related to various aspects of the present disclosure, which are described below. This disclosure is believed to be helpful in providing the reader with background information to facilitate a better understanding of the various aspects of the present disclosure. Accordingly, it should be understood that these statements are to be read in this light, and not just as an admissions of prior art.

Robotic assisted surgical systems have been adopted worldwide to gradually replace conventional surgical procedures such as open surgery and laparoscopic surgical procedures. The robotic assisted surgery offers various benefits to a patient during surgery and during post-surgery recovery time. The robotic assisted surgery equally offers numerous benefits to a surgeon in terms of enhancing the surgeon's ability to precisely perform surgery, less fatigue and a magnified clear three-dimensional (3D) vision of a surgical site. Further, in a robotic assisted surgery, the surgeon typically operates with a hand controller/master controller/surgeon input device/joystick at a surgeon console system to seamlessly receive and transfer complex actions performed by him/her giving the perception that he/she himself/herself is directly articulating a surgical tools/surgical instrument to perform the surgery. The surgeon operating on the surgeon console system may be located at a distance from a surgical site or may be located within an operating theatre where the patient is being operated on.

The robotic assisted surgical systems may comprise of multiple robotic arms aiding in conducting robotic assisted surgeries. The robotic assisted surgical system utilizes a sterile adapter/a sterile barrier to separate a non-sterile section of the multiple robotic arms from a mandatory sterile surgical tools/surgical instrument attached to one end of the multiple robotic arms. The sterile adaptor/sterile barrier may include a sterile plastic drape that envelops the multiple robotic arms and the sterile adaptor/sterile barrier that operably engages with the sterile surgical tools/surgical instrument in the sterile field.

During robotic assisted surgery, a trocar assembly is inserted into the tissue to create a port for surgical instruments access to the surgical site. The trocar assembly includes a cannula, an obturator, and a seal. The obturator facilitates in piercing the patient tissue so that the distal end of the cannula can be inserted through the pierced tissue. The seal is placed at the proximal end of the cannula to maintain the insufflation and prevent the CO2 leaking from the cannula insertion port. Plurality of cannula is inserted into the patient body through which the surgical instruments and endoscope system are inserted to access the surgical site. The multiple robotic arms after being draped may be brought close to the operating table for docking the arms to the respective cannula.

The main challenge with the existing cannula is that the distal end of the cannula is stiff and non-articulating due to which the articulating endoscope tip and articulating distal end of the surgical instruments are not compatible with the existing cannula.

In the light of aforementioned challenges, there is a need for articulating distal end of the cannula so as to accommodate the articulation of endoscope tip and articulating distal end of the surgical instruments.

### SUMMARY OF THE DISCLOSURE

Some or all of the above-mentioned problems related to providing training to the surgeons and OT staff are proposed to be addressed by certain embodiments of the present disclosure.

According to an aspect of the invention, there is disclosed an articulating cannula comprising a proximal end and a distal end. The distal end is configured to articulate between the first position and a second position. The articulating cannula further comprises a hollow tubular shaft connecting the proximal end and the distal end. The shaft being segmented into a first shaft and a second shaft, the first shaft and the second shaft secured by a flexible material. The proximal end comprises of a housing having a diameter substantially greater than the diameter of the shaft and configured to allow insertion of an articulating surgical instrument. The second shaft comprises of an outer layer and an inner layer of the flexible material with a compression spring in between. The second shaft is configured to facilitate the articulation of the distal end. The second shaft is further configured to prevent collapse of second shaft at the distal end. The spring is configured to maintain a constant cross section of the second shaft with or without articulation.

Optionally, the flexible material may be a flexible intermediate tube and the like.

Optionally, the first shaft and the second shaft are secured by a flexible material using at least one of an ultrasonic or over-mould type of sealing.

Optionally, the flexible material comprises at least one of Viton, Silicon, or natural rubber, etc.

Optionally, material utilized to make the spring comprises at least one of a Stainless steel, Music wire, or Plastic, etc.

Optionally, articulation of the distal end is in the range of 0-90° all round 360°.

Optionally, articulating surgical instrument may be an instrument with articulating tip associated with one or more surgical tasks, such as a forceps, a needle driver, a shears, a bipolar cauterizer, a tissue stabilizer or retractor, a clip applier, an anastomosis device, an imaging device, an endoscope, or an ultrasound probe, and the like.

Optionally, the spring helps to maintain remote center of motion (RCM) with patient body in a surgical robotic system.

Optionally, the spacing between the outer layer and the inner layer can be between 2.2 mm to 28 mm.

Optionally, the articulating endoscope comprises of a proximal end connected with a distal end by an endoscope shaft.

Other embodiments, systems, methods, apparatus aspects, and features of the invention will become apparent to those skilled in the art from the following detailed description, the accompanying drawings, and the appended claims. It will be appreciated that features of the present disclosure are susceptible to being combined in various combinations without departing from the scope of the present disclosure as defined by the appended claims.

### BRIEF DESCRIPTION OF THE DRAWINGS

The summary above, as well as the following detailed description of the disclosure, is better understood when read in conjunction with the appended drawings. For the purpose of illustrating the present disclosure, exemplary constructions of the disclosure are shown in the drawings. However, the present disclosure is not limited to specific methods and instrumentalities disclosed herein. Moreover, those skilled in the art will understand that the drawings are not to the scale. Wherever possible, like elements have been indicated by identical numbers.

Embodiments of the present disclosure will now be described, by way of example only, with reference to the following diagrams wherein:

FIG. 1 illustrates an example implementation of a multi arm teleoperated robotic surgical system which can be used with one or more features in accordance with an embodiment of the disclosure;

FIG. 2(a) illustrates a perspective view of a tool interface assembly mounted on a robotic arm in accordance with an embodiment of the invention;

FIG. 2(b) illustrates a perspective view of the tool interface assembly in accordance with an embodiment of the invention;

FIG. 3(a) illustrates a perspective view of a cannula in accordance with an embodiment of the disclosure;

FIG. 3(b) illustrates a perspective view of a cannula with distal end having layers and spring in between, in accordance with an embodiment of the disclosure; and

FIG. 3(c) illustrates a perspective view of a cannula along with an articulating endoscope in accordance with an embodiment of the disclosure.

### DETAILED DESCRIPTION OF THE DISCLOSURE

For the purpose of promoting an understanding of the principles of the disclosure, reference will now be made to the embodiment illustrated in the drawings and specific language will be used to describe the same. It will nevertheless be understood that no limitation of the scope of the disclosure is thereby intended, such alterations and further modifications in the illustrated system, and such further applications of the principles of the disclosure as illustrated therein being contemplated as would normally occur to one skilled in the art to which the disclosure relates.

It will be understood by those skilled in the art that the foregoing general description and the following detailed description are exemplary and explanatory of the disclosure and are not intended to be restrictive thereof. Throughout the patent specification, a convention employed is that in the appended drawings, like numerals denote like components.

Reference throughout this specification to “an embodiment”, “another embodiment”, “an implementation”, “another implementation” or similar language means that a particular feature, structure, or characteristic described in connection with the embodiment is included in at least one embodiment of the present disclosure. Thus, appearances of the phrase “in an embodiment”, “in another embodiment”, “in one implementation”, “in another implementation”, and similar language throughout this specification may, but do not necessarily, all refer to the same embodiment.

The terms “comprises”, “comprising”, or any other variations thereof, are intended to cover a non-exclusive inclusion, such that a process or method that comprises a list of steps does not include only those steps but may include other steps not expressly listed or inherent to such process or method. Similarly, one or more devices or sub-systems or elements or structures proceeded by “comprises . . . a” does not, without more constraints, preclude the existence of other devices or other sub-systems or other elements or other structures or additional devices or additional sub-systems or additional elements or additional structures.

Unless otherwise defined, all technical and scientific terms used herein have the same meaning as commonly understood by one of ordinary skill in the art to which this disclosure belongs. The device, system, and examples provided herein are illustrative only and not intended to be limiting.

The terms “a” and “an” herein do not denote a limitation of quantity, but rather denote the presence of at least one of the referenced items. Further, the term sterile barrier and sterile adapter denotes the same meaning and may be used interchangeably throughout the description.

Embodiments of the disclosure will be described below in detail with reference to the accompanying drawings.

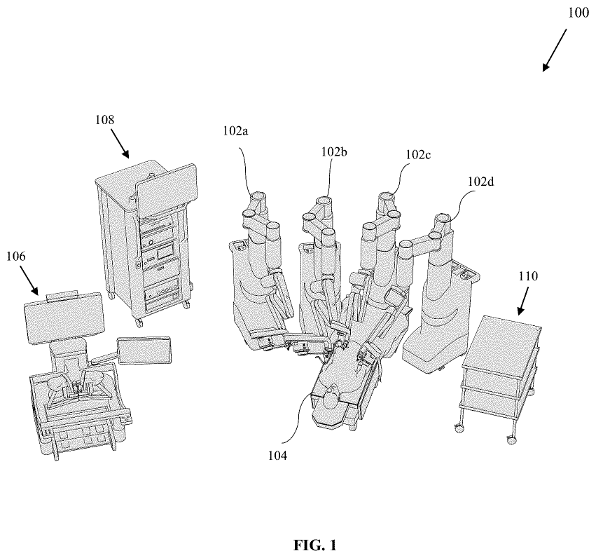

FIG. 1 illustrates an example implementation of a multi arm teleoperated robotic surgical system which can be used with one or more features in accordance with an embodiment of the disclosure. Specifically, FIG. 1 illustrates the multi arm robotic surgical system (100) having four robotic arms (102a), (102b), (102c), (102d) mounted on four robotic arm carts around an operating table (104). The four-robotic arms (102a), (102b), (102c), (102d) as depicted in FIG. 1 are for illustration purposes and the number of robotic arms may vary depending upon the type of surgery. The four robotic arms (102a), (102b), (102c), (102d) are arranged along the operating table (104) and may also be arranged in different manner but not limited to the robotic arms (102a), (102b), (102c), (1012d) arranged along the operating table (104). The robotic arms (102a), (102b), (102c), (102d) may be separately mounted on the four robotic arm carts or the robotic arms (102a), (102b), (102c), (102d) mechanically and/or electronically connected with each other or the robotic arms (102a), (102b), (102c), (102d) connected to a central body (not shown) such that the robotic arms (102a), (102b), (102c), (102d) branch out of a central body (not shown). Further, the multi arm teleoperated robotic surgical system (100) may include a surgeon console system (106), a vision cart (108), and an accessory table (110) for surgical instruments.

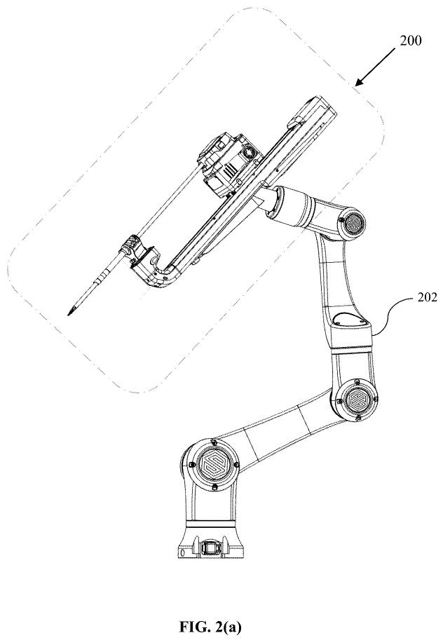

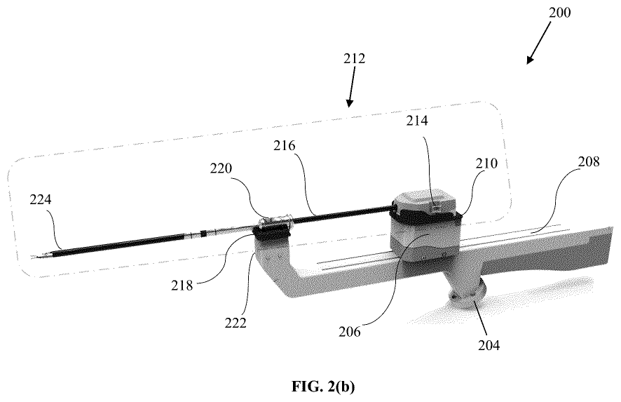

FIG. 2(a) illustrates a perspective view of a tool interface assembly mounted on a robotic arm in accordance with an embodiment of the invention. The tool interface assembly (200) is mounted on the robotic arm (202) of the robotic surgical system (100) (shown in FIG. 1). The tool interface assembly (200) is the main component for performing the robotic surgery on a patient. The robotic arm (202) as shown in FIG. 2(a) is for the illustration purpose only and other robotic arms with different configurations, degree of freedom (DOF) and shapes may be used.

FIG. 2(b) illustrates a perspective view of the tool interface assembly. The tool interface assembly (200), as depicted in the FIG. 2(b), comprises of an ATI (arm and tool interface) connector (204) which facilitates the tool interface assembly (200) to operationally connect with the robotic arm (shown in FIG. 2(a)). Further, the tool interface assembly (200) further comprises of an actuator assembly (206) mounted on a guiding mechanism and capable of linearly moving along the guiding mechanism. The guiding mechanism depicted in FIG. 2(b) is a guide rail (208). The movement of the actuator assembly (206) along the guide rail (208) is controlled by the surgeon with the help of controllers on the surgeon console system (106) as shown in FIG. 1. A sterile adapter assembly (210) is releasably mounted on the actuator assembly (206) to separate a non-sterile part of the robotic arm from a sterile surgical tool assembly (212). A locking mechanism (not shown) is provided to releasably lock and unlock the sterile adapter assembly (210) with the actuator assembly (206). The sterile adapter assembly (210) detachably engages from the actuator assembly (206) which drives and controls the sterile surgical instrument in a sterile field. In another embodiment, the surgical tool assembly (212) also may releasably lock/unlock or engage/disengage with the sterile adapter assembly (210) by means of a push button (214).

The surgical tool assembly (212) includes a shaft (216) and end effector (224). The end effector (224) may comprise of a surgical instrument or may be configured to attach a surgical instrument. Further, the end effector (224) may include a functional mechanical degree of freedom, such as jaws that open or close, or a knife that translates along a path. The surgical tool assembly (212) may also contain stored (e.g., on a semiconductor memory inside the instrument) information that may be permanent or may be updatable by the robotic surgical system (100).

A cannula gripper (218) is provided on the tool interface assembly (200) and is configured to grip a cannula (220) which receives the shaft (216) through an opening (not shown). The cannula gripper (218) is detachably attached to one end of the tool interface assembly (200). Alternatively, the cannula gripper (218) may have a circular body for receiving the cannula (220) and comprise of grooves (not shown) to grip the cannula (220) at a stationary position. The cannula gripper (218) may be affixed at a mount (222) of the tool interface assembly (200) and may be configured to grip or secure the cannula (220) such that cannula (220) is stable while performing surgical operations.

The end effector (224) may be a surgical instrument like a forceps, a needle, etc., associated with one or more surgical tasks or an endoscope/ultrasound probe and the like. Some surgical instruments further provide an articulated support (sometimes referred to as a “wrist”) for the surgical tool assembly (212) such that the position and orientation of the surgical tool assembly (212) may be manipulated with one or more mechanical degrees of freedom in relation to the shaft (216). The surgical instrument may have articulating tip.

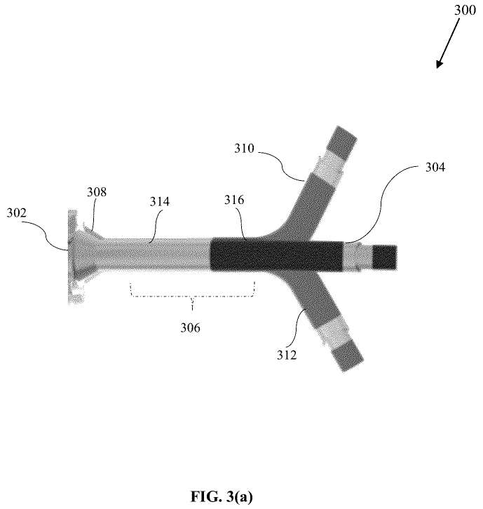

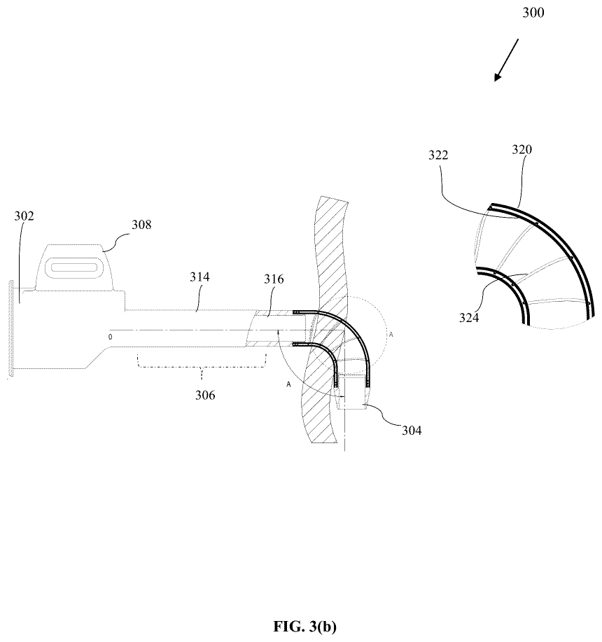

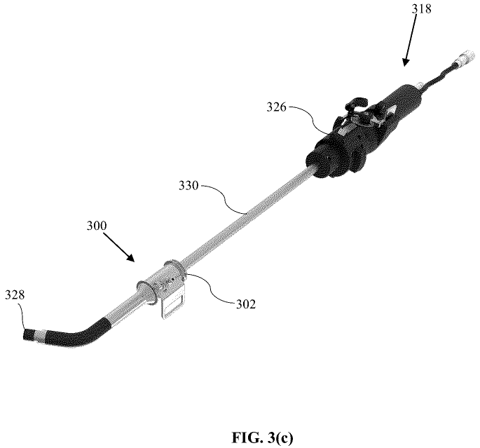

FIG. 3(a) and FIG. 3(b) illustrate a perspective view of a cannula in accordance with an embodiment of the disclosure. As illustrated in FIG. 3(a) and FIG. 3(b), an articulating cannula (300) comprises of a proximal end (302) and a distal end (304). A hollow tubular shaft (306) connects the proximal end (302) and the distal end (304) of the cannula (300). The proximal end (302) comprises of a housing (308) having a diameter substantially greater than the diameter of the hollow shaft (306) of the cannula (300). The proximal end (302) is configured to allow insertion of a surgical instrument. The distal end (304) of the cannula (300) is capable of articulating in a first position (310) and a second position (312).

According to an embodiment, the hollow tubular shaft (306) of the articulating cannula (300) is segmented in two parts, i.e., first shaft (314) and a second shaft (316). The first shaft (314) and the second shaft (316) are secured by a flexible material using at least one of an ultrasonic or over-mould type of sealing. The flexible material comprises at least one of Viton, Silicon, or natural rubber, etc. The flexible material may also be a flexible intermediate tube and the like. As illustrated in FIG. 3(b), the second shaft (316) comprises of an outer layer (320) and an inner layer (322) of the flexible material with a compression spring (324) in between.

According to an embodiment, the material utilized to make the spring (324) comprises at least one of a Stainless steel, Music wire, or Plastic, etc. According to a specific advantageous embodiment, the spacing between the outer layer (320) and the inner layer (322) can be between 2.2 mm to 28 mm. The spring (324) is configured to maintain a constant cross section of the second shaft (316) with or without articulation. The second shaft (316) is configured to facilitate the articulation of the distal end (304). The second shaft (316) is further configured to prevent collapse of second shaft (316) at the distal end (304). The articulation of the distal end (304) is in the range of 0-90° all round 360°.

FIG. 3(c) illustrates a perspective view of an articulating cannula along with an articulating endoscope in accordance with an embodiment of the disclosure. As illustrated in FIG. 3(c), an endoscope (318) is inserted through the proximal end (302) of the cannula (300). The endoscope (318) comprises of a proximal end (326) and a distal end (328). An endoscope shaft (330) connects the proximal end (326) and the distal end (328) of the endoscope (318). The endoscope shaft (330) at its distal end (328) is capable of articulating in different directions. The articulating surgical instrument (318) may be a surgical instrument with articulating tip associated with one or more surgical tasks, such as a forceps, a needle driver, a shears, a bipolar cauterizer, a tissue stabilizer or retractor, a clip applier, an anastomosis device, an imaging device, an endoscope, or an ultrasound probe, and the like. The spring (324) helps to maintain remote center of motion (RCM) with patient body in a surgical robotic system.

The major advantage of the proposed articulating cannula is that it provides articulation of distal end ranging between 0-90° all round 360°. Further, another advantage of the proposed articulating cannula is that the cross section of the shaft is maintained constant with or without articulation. Also, the collapse of distal end is prevented while articulation of distal end of the cannula.

The foregoing description of exemplary embodiments, of the present disclosure has been presented for purposes of illustration and description. They are not intended to be exhaustive or to limit the disclosure to the precise forms disclosed, and obviously many modifications and variations are possible in light of the above teaching. The exemplary embodiment was chosen and described in order to best explain the principles of the disclosure and its practical application, to thereby enable others skilled in the art to best utilize the disclosure and various embodiments with various modifications as are suited to the particular use contemplated. It is understood that various omissions, substitutions of equivalents are contemplated as circumstance may suggest or render expedient but is intended to cover the application or implementation without departing from the spirit or scope of the claims of the present disclosure.

Benefits, other advantages, and solutions to problems have been described above with regard to specific embodiments. However, the benefits, advantages, solutions to problems, and any component(s) that may cause any benefit, advantage, or solution to occur or become more pronounced are not to be construed as a critical, required, or essential feature or component of any or all the claims.

While specific language has been used to describe the disclosure, any limitations arising on account of the same are not intended. As would be apparent to a person in the art, various working modifications may be made to the apparatus in order to implement the inventive concept as taught herein.

1. An articulating cannula (300) comprising:
a proximal end (302) and a distal end (304), the distal end (304) configured to articulate between a first position (310) and a second position (312); and
a hollow tubular shaft (306) connecting the proximal end (302) and the distal end (304), the shaft (306) being segmented into a first shaft (314) and a second shaft (316), the first shaft (314) and the second shaft (316) secured by a flexible material,
wherein the proximal end (302) comprises of a housing (308) having diameter substantially greater than the diameter of the shaft (306), and configured to allow insertion of an articulating surgical instrument (318),
wherein the second shaft (316) comprises of an outer layer (320) and an inner layer (322) of the flexible material with a compression spring (324) in between, the second shaft (316) configured to facilitate the articulation of the distal end (304),
wherein the second shaft (316) further configured to prevent collapse of second shaft (316) at the distal end (304),
wherein the spring (324) is configured to maintain a constant cross section of the second shaft (316) with or without articulation.

2. The articulating cannula (300) as claimed in claim 1, wherein the flexible material may be a flexible intermediate tube and the like.

3. The articulating cannula (300) as claimed in claim 1, wherein the first shaft (314) and the second shaft (316) are secured by a flexible material using at least one of an ultrasonic or over-mould type of sealing.

4. The articulating cannula (300) as claimed in claim 1, wherein the flexible material comprises at least one of Viton, Silicon, or natural rubber, etc.

5. The articulating cannula (300) as claimed in claim 1, wherein material utilized to make the spring (324) comprises at least one of a Stainless steel, Music wire, or Plastic, etc.

6. The articulating cannula (300) as claimed in claim 1, wherein articulation of the distal end (304) is in the range of 0-90° all round 360°.

7. The articulating cannula (300) as claimed in claim 1, wherein articulating surgical instrument (318) may be an instrument with articulating tip associated with one or more surgical tasks, such as a forceps, a needle driver, a shears, a bipolar cauterizer, a tissue stabilizer or retractor, a clip applier, an anastomosis device, an imaging device, an endoscope, or an ultrasound probe, and the like.

8. The articulating cannula (300) as claimed in claim 1, wherein the spring (324) helps to maintain remote center of motion (RCM) with patient body in a surgical robotic system.

9. The articulating cannula (300) as claimed in claim 1, wherein spacing between the outer layer (320) and the inner layer (322) can be between 2.2 mm to 28 mm.

10. The articulating cannula (300) as claimed in claim 1, wherein the articulating endoscope (318) comprises of a proximal end (326) connected with a distal end (328) by an endoscope shaft (330).
